(19) United States
(12) Patent Application Publication
(10) Pub. No.: US 2025/0345212 A1
(43) Pub. Date: Nov. 13, 2025

(54) URINE TESTING GARMENT

(71) Applicant: Barbara Rehling, Festus, MO (US)

(72) Inventor: Barbara Rehling, Festus, MO (US)

(21) Appl. No.: 18/659,884

(22) Filed: May 9, 2024

(51) Int. Cl. A61F 13/42; A61F 13/53; A61F 13/80

(52) U.S. Cl. CPC A61F 13/42; A61F 13/53; A61F 13/80; A61F 2013/530007

(57) ABSTRACT

A urine testing apparatus for testing urine without catheterization includes a garment shaped and configured for being worn over a crotch of a user. The garment has a crotch-covering portion which extends from a front end to a rear end. An indicator panel is mounted to the crotch-covering portion of the garment, the indicator panel comprises a plurality of indicator sections configured to indicate data corresponding to urine contacting the indicator section. The indicator may indicate the presence of various substances in the urine and qualities of the urine such as acidity.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to urine testing apparatuses and more particularly pertains to a new urine testing apparatus for testing urine without catheterization.

### (2) Description of Related Art Including Information Disclosed Under 37 CFR 1.97 and 1.98

It is often necessary to test the urine of an individual for markers that indicate a urinary tract infection or other medical condition. Particularly for elderly individuals, symptoms of urinary tract infections may be vague, making completion of a urine test essential to diagnosis and treatment. Further, incontinence is one prevalent symptom of urinary tract infections in elderly people, which also presents a problem for collecting a urine sample. Often, the patient is subjected to an uncomfortable catheterization process for urine collection. These problems present a need for a more comfortable urine collection apparatus which may be used for incontinent individuals.

The prior art discloses garments with chemical reagents for detecting various substances in the urine of the wearers of the garments. These garments make it possible to collect urine from incontinent individuals by simply reacting to urine that is released while the individuals wear such garments. However, the prior art fails to describe such an apparatus which arranges various testing sections in front-to-back extending strips to ensure that each section is able to contact the urine. The prior art also fails to describe testing sections which comprise substrates of silica gel or crystals.

### BRIEF SUMMARY OF THE INVENTION

An embodiment of the disclosure meets the needs presented above by generally comprising a garment shaped and configured for being worn over a crotch of a user. The garment has a crotch-covering portion which extends from a front end to a rear end. An indicator panel is mounted to the crotch-covering portion of the garment, the indicator panel comprises a plurality of indicator sections configured to indicate data corresponding to urine contacting the indicator section.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a urine testing apparatus according to an embodiment of the disclosure.

FIG. 2 is a top view of an embodiment of the disclosure.

FIG. 3 is a cross-section view of an embodiment of the disclosure taken from Line 3-3 in FIG. 2.

FIG. 4 is a detail view of an embodiment of the disclosure taken from Window 4 in FIG. 2.

### DETAILED DESCRIPTION OF THE INVENTION

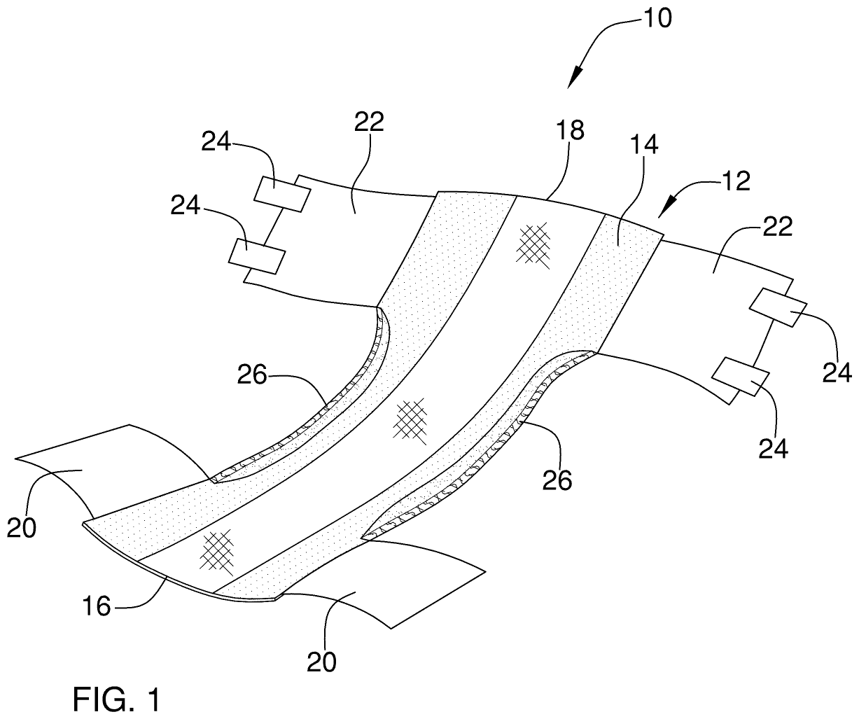

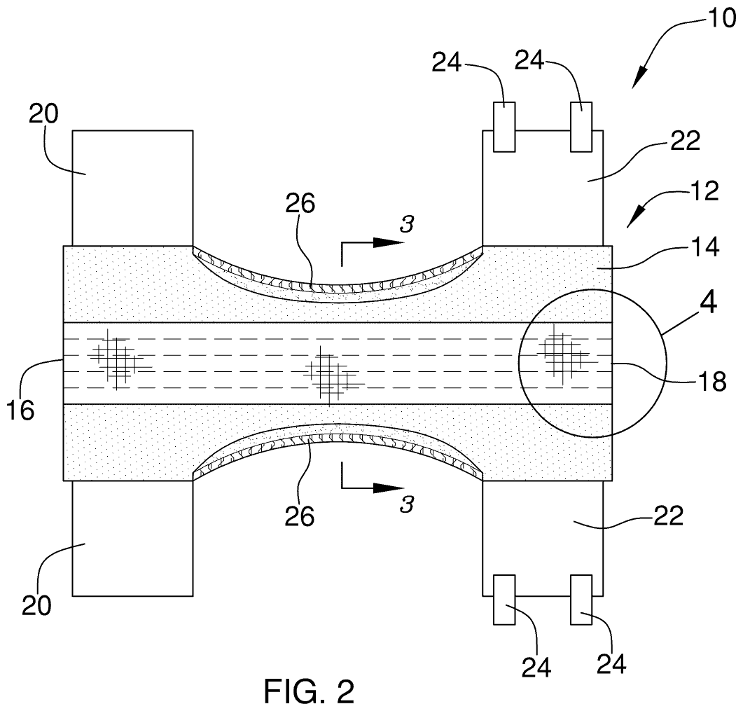

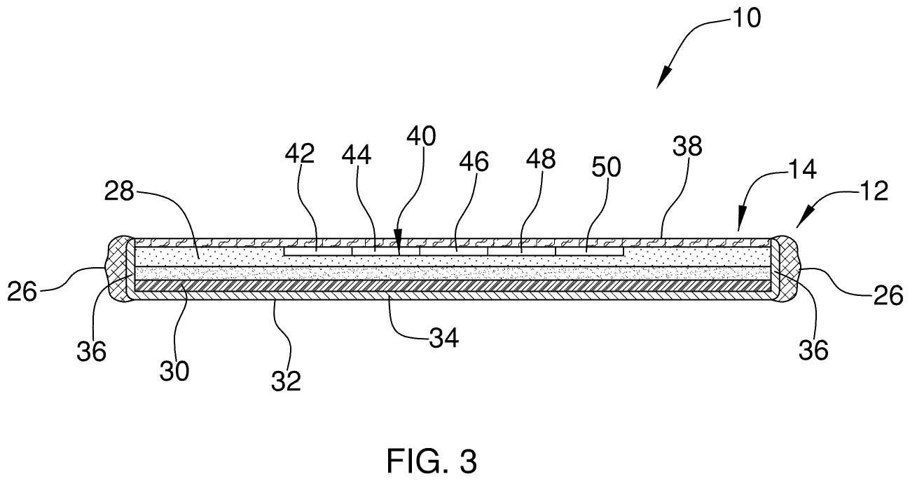

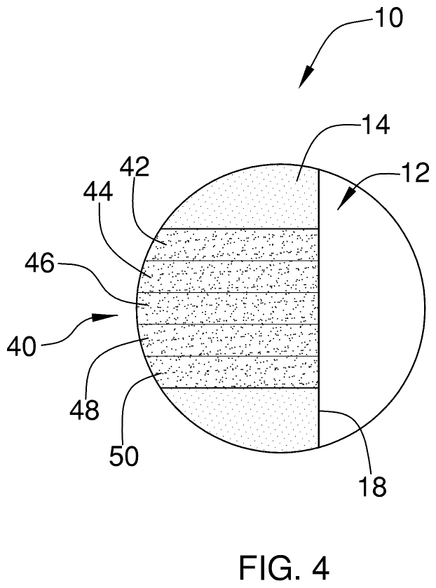

With reference now to the drawings, and in particular to FIGS. 1 through 4 thereof, a new urine testing apparatus embodying the principles and concepts of an embodiment of the disclosure and generally designated by the reference numeral 10 will be described.

As best illustrated in FIGS. 1 through 4, the urine testing apparatus 10 generally comprises a garment 12 shaped and configured for being worn over a crotch of a user. The garment 12 defines a diaper with a crotch-covering portion 14 that extends from a front end 16 to a rear end 18. A pair of front straps 20 are coupled to the crotch-covering portion 14 near the front end 16 and extend in opposite lateral directions from the crotch-covering portion 14. A pair of rear straps 22 are coupled to the crotch-covering portion 14 near the rear end 18 and extend in opposite lateral directions from the crotch-covering portion 14. The front straps 20 are attachable to the rear straps 22 via adhesive connectors 24 to retain the garment 12 on the user. The garment 12 also includes stretchable members 26 which extend along lateral edges of the crotch-covering portion 14. When the garment 12 is worn, the stretchable members 26 stretch to accommodate placement of the user's legs and act to seal against the user's legs. The stretchable members 26 may comprise an elastic material such as rubber, silicone, or the like. In other embodiments, the garment 12 may define a brief, a pair of shorts, or the like. In yet other embodiments, the garment 12 may be an insert which is placed into another article of clothing to be retained over the user's crotch during use.

The crotch-covering portion 14 comprises a plurality of layers, including an absorbent layer 28, a waterproof layer 30, a breathable layer 32, and a permeable layer 38. The absorbent layer 28 is configured to absorb urine and to inhibit leakage of the urine from the absorbent layer 28. The absorbent layer 28 may comprise sodium polyacrylate, fluff pulp, or any other suitable material. The waterproof layer 30 is positioned under the absorbent layer 28. The waterproof layer 30 may comprise any suitable liquid-impermeable material such as vinyl, polyethylene, polypropylene, or the like.

The breathable layer 32 has a base portion 34 positioned under the waterproof layer 30 and a pair of side portions 36 which are coupled to the base portion 34 and extend upwardly from the base portion 34 past each of the waterproof layer 30 and the absorbent layer 28 to a top side of the crotch-covering portion 14. The breathable layer 32 comprises breathable and liquid-impermeable material such as polyurethane laminate or the like. It is noted that as used in the present disclosure, a “breathable” material is one which facilitates passage of a moisture vapor through the material, and such materials may also prevent passage of the same substance constituting the moisture vapor when it is in a liquid state. The permeable layer 38 is positioned atop the absorbent layer 28 and is liquid-permeable. The permeable layer 38 may comprise any fabric or other flexible material with an open knit, open weave, or otherwise porous structure to facilitate passage of urine therethrough.

An indicator panel 40 is mounted to the crotch-covering portion 14 of the garment 12 between the absorbent layer 28 and the permeable layer 38. The permeable layer 38 protects the indicator layer from damage from wear while allowing urine to pass through to the indicator layer. The indicator panel 40 comprises a plurality of indicator sections configured to indicate data corresponding to urine contacting the indicator section. The indicator sections may indicate the data via a chemical change of pigment or any other suitable means. Each indicator section extends from the front end 16 of the crotch-covering portion 14 to the rear end 18. Each indicator section comprises a substrate and an indicator reagent. The substrate of each indicator section comprises silica, which may be in a gel or crystalline form.

The plurality of indicator sections includes a nitrite section 42, a bacteria section 44, a blood section 46, a pH section 48, and a leukocyte section 50. The nitrite section 42 is configured to indicate a presence of nitrites in the urine when the urine comes into contact with the nitrite section 42. The nitrite section 42 may detect nitrite by using the Griess reaction in which the nitrite reacts in an acid medium with an aromatic amine (para-arsanilic acid or sulphanilamide) in order to form a diazonium salt that in turn reacts with tetrahydrobenzoquinoline to produce a pink azo dye. Other suitable compositions which facilitate the identification of nitrites in the urine also may be used.

The bacteria section 44 configured to indicate a presence of Gram positive and Gram negative bacteria. The bacteria section 44 may comprise reagents of crystal violet, iodine, alcohol, and safranin in sufficient arrangement to facilitate a Gram staining procedure as known in the prior art to identify Gram positive and Gram negative bacteria. Other reagents may be used, including substituting alcohol with acetone and substituting the safranin with carbol fuchsin. The blood section 46 is configured to indicate a presence of blood. The reagent of the blood section 46 may be diisopropylbenzene dihydroperoxide or any other suitable reagent. The pH section 48 is configured to indicate a pH of the urine, and the reagent of the pH section 48 may be bromothymol blue or any other suitable indicator. The leukocyte section 50 is configured to indicate a presence of leukocytes, and identification of leukocytes may comprise an action of leukocyte esterase in catalysing the hydrolysis of an ester of indolecarboxylic acid. The indoxyl that is liberated from this process combines with a diazonium salt in order to produce a violet colored azole dye. Other suitable compositions which facilitate the identification of leukocytes also may be used. The plurality of indicator sections may include other sections for detecting the presence of proteins, glucose, or other substances or conditions in the urine.

In use, the user wears the garment 12. When the user urinates, either intentionally or due to incontinence, the urine contacts the plurality of indicator sections of the indicator panel 40. As described more fully above, various substances present in the urine react with the reagents of the plurality of indicator sections to indicate the presence of the various substances or to indicate a quality of the urine, such as the acidity/alkalinity as described by a pH indication. The urine is then collected and retained in the absorbent layer 28 of the garment 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A urine testing apparatus comprising:
a garment shaped and configured for being worn over a crotch of a user, the garment having a crotch-covering portion which extends from a front end to a rear end; and
an indicator panel mounted to the crotch-covering portion of the garment, the indicator panel comprising a plurality of indicator sections configured to indicate data corresponding to urine contacting the indicator section.

2. The apparatus of claim 1, wherein the crotch-covering portion of the garment comprises an absorbent layer configured to absorb urine.

3. The apparatus of claim 2, wherein the absorbent layer is configured to lock in urine to inhibit leakage of the urine from the absorbent layer.

4. The apparatus of claim 3, wherein the absorbent layer comprises sodium polyacrylate.

5. The apparatus of claim 3, wherein the absorbent layer comprises fluff pulp.

6. The apparatus of claim 2, wherein the crotch-covering portion of the garment further comprises a waterproof layer positioned under the absorbent layer, the waterproof layer being liquid-impermeable.

7. The apparatus of claim 6, wherein the crotch-covering portion of the garment further comprises a breathable layer positioned under the waterproof layer, the breathable layer being breathable and liquid-impermeable.

8. The apparatus of claim 7, wherein the breathable layer has a base portion and a pair of side portions, the base portion being positioned adjacent to the waterproof layer, the side portions extending from the base portion past each of the waterproof layer and the absorbent layer to a top side of the crotch-covering portion.

9. The apparatus of claim 2, wherein the crotch-covering portion of the garment further comprises a permeable layer positioned atop the absorbent layer, the permeable layer being liquid-permeable.

10. The apparatus of claim 9, wherein the indicator panel is positioned between the absorbent layer of the crotch-covering portion and the permeable layer.

11. The apparatus of claim 1, wherein each indicator section extends from the front end of the crotch-covering portion to the rear end.

12. The apparatus of claim 1, wherein each indicator section comprises a substrate and an indicator reagent.

13. The apparatus of claim 12, wherein the substrate of each indicator section comprises silica.

14. The apparatus of claim 1, wherein the plurality of indicator sections includes a nitrite section configured to indicate a presence of nitrites.

15. The apparatus of claim 1, wherein the plurality of indicator sections includes a bacteria section configured to indicate a presence of Gram positive and Gram negative bacteria.

16. The apparatus of claim 1, wherein the plurality of indicator sections includes a blood section configured to indicate a presence of blood.

17. The apparatus of claim 1, wherein the plurality of indicator sections includes a pH section configured to indicate a pH of the urine.

18. The apparatus of claim 1, wherein the plurality of indicator sections includes a leukocyte section configured to indicate a presence of leukocytes.

19. A urine testing apparatus comprising:
a garment shaped and configured for being worn over a crotch of a user, the garment having a crotch-covering portion which extends from a front end to a rear end, the crotch-covering portion comprising a plurality of layers, the plurality of layers including:
an absorbent layer configured to absorb urine, the absorbent layer being configured to lock in urine to inhibit leakage of the urine from the absorbent layer, the absorbent layer comprising sodium polyacrylate;
a waterproof layer positioned under the absorbent layer, the waterproof layer being liquid-impermeable;
a breathable layer positioned under the waterproof layer, the breathable layer being breathable and liquid-impermeable, the breathable layer having a base portion and a pair of side portions, the base portion being positioned adjacent to the waterproof layer, the side portions extending from the base portion past each of the waterproof layer and the absorbent layer to a top side of the crotch-covering portion; and
a permeable layer positioned atop the absorbent layer, the permeable layer being liquid-permeable; and
an indicator panel mounted to the crotch-covering portion of the garment, the indicator panel comprising a plurality of indicator sections configured to indicate data corresponding to urine contacting the indicator section, the indicator panel being positioned between the absorbent layer of the crotch-covering portion and the permeable layer, each indicator section extending from the front end of the crotch-covering portion to the rear end, each indicator section comprising a substrate and an indicator reagent, the substrate of each indicator section comprising silica, the plurality of indicator sections including:
a nitrite section configured to indicate a presence of nitrites;
a bacteria section configured to indicate a presence of Gram positive and Gram negative bacteria;
a blood section configured to indicate a presence of blood;
a pH section configured to indicate a pH of the urine; and
a leukocyte section configured to indicate a presence of leukocytes.
